(19) United States
(12) Patent Application Publication
(10) Pub. No.: US 2006/0161066 A1
(43) Pub. Date: Jul. 20, 2006

(54) Feature-based editing for electrocardiography

(75) Inventor: Jonathan L. Elion, Wakefield, RI (US)

Correspondence Address: HOUSTON ELISEEVA, 4 MILITIA DRIVE, SUITE 4, LEXINGTON, MA, 02421, US

(73) Assignee: Heartlab, Inc., Westerly, RI (US)

(21) Appl. No.: 11/335,860

(22) Filed: Jan. 18, 2006

Related U.S. Application Data
(60) Provisional application No. 60/644,888, filed on Jan. 18, 2005.

(51) Int. Cl. A61B 5/04

(52) U.S. Cl. 600/509

(57) ABSTRACT

A method and system for generating electrocardiogram reports allows for the editing of features in the electrocardiogram interpretation process. This improves the accuracy of machine interpretation of the ECG data thereby facilitating the analysis and generation of the final report by the physician. The present method and system are most useful in host-based ECG interpretation systems where the physician accesses the ECG data at a workstation including a machine interpretation that is generated typically at the ECG cart. The physician is then provided with the ability to modify the features in the ECG data and generate a new host-based interpretation based on the original ECG data and the features specified by the physician.

### RELATED APPLICATIONS

This application claims the benefit under 35 USC 119(e) of U.S. Provisional Application No. 60/644,888, filed on Jan. 18, 2005, which is incorporated herein by reference in its entirety.

### BACKGROUND OF THE INVENTION

Electrocardiography is a technology for the detection and diagnosis of cardiac conditions. An electrocardiograph is a medical device capable of recording the potential differences generated by the electrical activity of the heart. An electrocardiogram (ECG or EKG) is produced by the electrocardiograph. It typically comprises the ECG wave data that describes the heart's electrical activity as a function of time.

The heart's electrical activity is detected by sensing electrical potentials via a series of electrode leads that are placed on the patient at defined locations on the patient's chest and limbs. Systems with ten (10) separate ECG leads and digital data capture/storage are typical. During electrocardiography, the detected electrical potentials are recorded and graphed as ECG wave data that characterize the depolarization and repolarization of the cardiac muscle.

ECG interpretation is performed by analyzing the various cardiac electrical events presented in the ECG wave data. Generally, the ECG wave data comprise a P wave, which indicates atrial depolarization, a QRS complex, which represents ventricular depolarization, and a T-wave representing ventricular repolarization.

State-of-the-art ECG systems provide for the machine interpretation of the ECG data. These systems are designed to measure features of the ECG wave data from the patient. The various features of portions of the ECG, such as intervals, segments and complexes, including their amplitude, direction, and duration of the waves and their morphological aspects, are measured. Then all of the feature information is analyzed together. From this feature information, these systems are able to generate machine ECG interpretations diagnosing normal and abnormal cardiac rhythms and conduction patterns. These interpretations are often used by the physician/cardiologist as the basis of an ECG report for a given patient.

### SUMMARY OF THE INVENTION

With advances in technology, including more accurate ECG machines and increased sophistication in the software interpretation algorithms, machine-generated ECG interpretations have become increasingly accurate. Despite this trend, however, a nontrivial number of these machine-generated ECG interpretations will be incorrect. And, these incorrect ECG interpretations often represent a frustration to the physician/cardiologic, since the physician will be forced to function in a clerical role using text-editing tools to correct the machine-generated interpretations in the process of drafting the patient's ECG report.

Even though algorithms for computer-based ECG interpretation are generally quite accurate, errors are commonly made by the computer in making measurements and determining the low-level and high-level features in the first place. When these are made available for review and correction, the cardiologist can interact at a higher level of sophistication, acting more as a domain expert and less like a clerk or secretary. In addition, all of the internal checks for mutually exclusive conditions can remain in place (done during the final generation step of the interpretation process), thereby greatly reducing inadvertent errors and/or inconsistencies.

The present invention is directed to a method and system for generating electrocardiogram reports. It allows for the editing of features in the electrocardiogram interpretation process. This improves the accuracy of machine interpretation of the ECG data thereby facilitating the analysis and generation of the final report by the physician.

The present method and system are most useful in host-based ECG interpretation systems where the physician accesses the ECG data at a workstation including a machine interpretation that is generated typically by the host system or workstation. The physician is then provided with the ability to modify the features in the ECG data and generate a new host-based interpretation based on the original ECG data and the features specified by the physician.

In general, according to one aspect, the invention features a method for generating an electrocardiogram report. This method comprises receiving ECG data at a physician workstation and enabling the physician to review, specify, and, if needed, correct the values for features of the ECG data. An interpretation of the ECG data is then generated based on the values of the features specified by the physician. Finally, the interpretation is stored after physician editing in a patient database.

In the typical implementation, the ECG data include the ECG wave data and an ECG machine interpretation of that ECG wave data. Typically, the ECG wave data are generated at an ECG cart that is operated by a nurse or technician and the interpretation is generated by the host system.

Then, in the preferred interpretation process, the physician selects whether to edit the final host machine interpretation and finalize the report or specify new values for the features. Typically, the physician will select to edit the final machine interpretation if it is determined to be basically or largely accurate after having reviewed the ECG wave data.

In the typical implementation, the step of enabling feature value specification comprises presenting machine-generated values for features of the ECG wave data and enabling the physician to specify different values for at least one of the features. Then, the final steps of the host-based interpretation are re-performed in which a new interpretation of the ECG is generated using the specified values for the features from the physician and the machine-generated values for other features.

In general according to another aspect, the invention features a system for generating an electrocardiogram (ECG) report. The system comprises a workstation that receives ECG data and enables specification of values for features of the ECG data and an interpretation system for evaluating of the ECG data based on the values of the features specified by a user and generating an interpretation for storage in a patient database. typically the interpretation system runs on the workstation or host system.

In general according to another aspect, the invention features a computer software product for generating an electrocardiogram (ECG) report. The product comprises a computer-readable medium, such as a compact disk, in which program instructions are stored. These instructions, when read by a computer, cause the computer to receive ECG data at a physician workstation, and enable a user to specify values for features of the ECG data. The instructions also provide for the generation of an interpretation of the ECG data based on the values of the features specified by the user, and store the interpretation after possible editing in a patient database.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram illustrating the electrocardiogram (ECG) workflow in a typical hospital;

FIG. 2 is a flow diagram illustrating the machine interpretation process in a conventional ECG device or host-based interpretation system;

FIG. 3 shows prototypical ECG wave data illustrating the various portions of the wave;

FIG. 4 shows a conventional interface in an ECG report editing system;

FIG. 5 shows a series of text statements as would be generated by machine interpretation for an exemplary ECG report as is conventional; and

FIG. 6 is a flow diagram illustrating the process for ECG feature specification and ECG report generation according to the present invention.

### DETAILED DESCRIPTION OF THE PREFERRED EMBODIMENTS

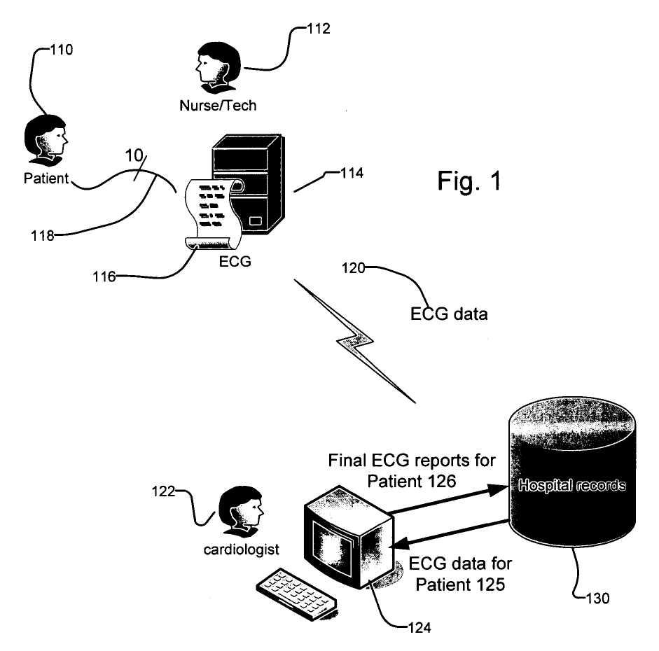

FIG. 1 illustrates the electrocardiogram (ECG) workflow in a typical hospital.

A nurse or ECG technician 112 interacts with the patient 110 to acquire the ECG data. In many modem systems, the ECG machine 114 is an ECG cart that is moved throughout the hospital between patient, examining, and operating rooms.

In operation, the ten (10) leads 118 of the ECG device 114 are placed on the limbs and torso of the patient 110. Then, a printout of the ECG wave data 116 is generated at the cart using twelve (12) combinations of the leads that have been placed on the patient. Also, ECG data 120 including the wave data, identifying information about the patient, and possibly the machine-generated ECG interpretation are digitally stored in the ECG cart 114 and/or transmitted to a central hospital records data storage and host system 130.

The present invention generally applies to a host-based interpretation and editing systems. In these systems, a cardiologist 122 accesses the ECG data 125 from the records database and host system 130 via a workstation 124. These hospital records will store preliminary ECG data and machine interpretations and the subsequent final reports that are the product of the editing process by the cardiologist 122 at the workstation 124. The final reports will then be entered into the patients' records.

The workstation 124 is provided with standard software for accessing and editing the ECG data and machine-generated interpretations reports from the host system 130 and generating the final cardiologist-reviewed ECG reports 126.

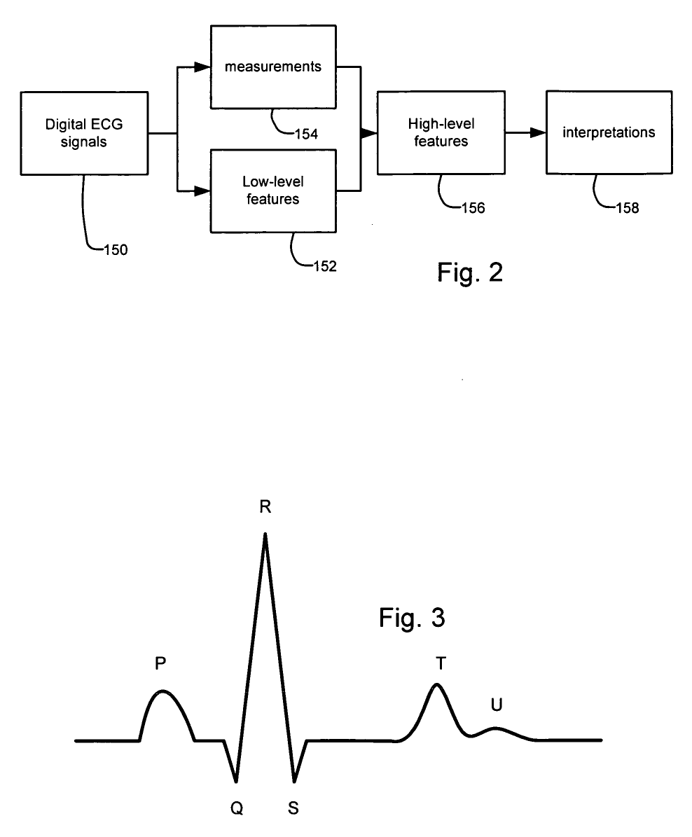

FIG. 2 illustrates the general process by which these machine interpretations are generated by the host system 130 or possibly by the workstation 124.

Specifically, the digital ECG signals or wave data 150 are acquired in step 150 and stored such as by the ECG cart 114. Measurements of portions of this ECG wave data are made in step 154 and low-level features 152 are typical identified in the wave data at the host system 130. This information is then combined in step 156 when high-level features are determined. Based on these calculated features, the final interpretation is generated in step 158.

The features typically relate to the length and amplitude of the various components of a selected ECG wave from one typical cardiac cycle out of the usually very long wave data set that the machine acquires. In other cases, an average ECG wave is calculated from a series of waves to form the basis of the interpretation. Features can be identified for each of the individual 12 leads, or combined to derive features of the overall ECG.

FIG. 3 illustrates a prototypical ECG wave. It generally comprises a P wave, a QRS wave complex, a T-wave, and a U wave. The features that the typical system uses can be dependent on specific characteristics of that system but will include intervals, segments and complexes, including amplitude, direction, and duration of the waves and their morphological aspects.

Table I below lists a number of exemplary features and feature information that are generated in order to enable the subsequent machine ECG interpretation.

TABLE

| Group Label | Features |
|---|---|
| RR intervals | Pre-RR interval |
| Time duration | Post-RR interval |
| between two | Average RR-interval |
| consecutive R | Local avg. RR-interval |
| waves of the ECG | |
| Heart-beat | QRS duration (QRS offset − QRS onset) of |
| Intervals A [B] | lead A [B] |
| | T-wave duration (T-wave offset − QRS |
| | offset) of lead A [B] |
| | P wave flag for lead A [B] |
| Morphology | ECG morphology between QRS onset |
| 1A [1B] | and QRS offset |
| | ECG morphology between QRS offset |
| | and T-wave offset |
| Morphology | Normalized ECG morphology between |
| 2A [2B] | QRS onset and QRS offset |
| | Normalized ECG morphology between |
| | QRS offset and T-wave offset |
| Morphology | ECG morphology between FP − 50 ms to |
| 3A [3B] | FP + 100 ms] |
| | ECG morphology between FP + 150 ms to |
| | FP + 500 ms |
| Morphology | Normalized ECG morphology between |
| 4A [4B] | FP − 50 ms to FP + 100 ms |
| | Normalized ECG morphology between |
| | FP + 150 ms to FP + 500 ms |

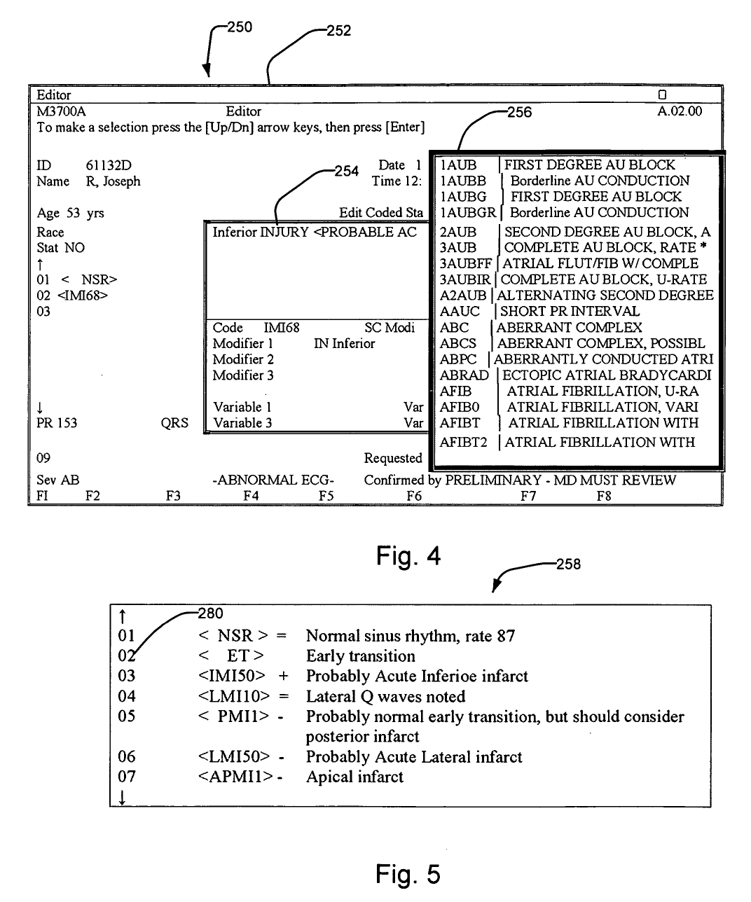

FIG. 4 illustrates a typical interface 250 for an ECG report editor running on workstation 124. In the specific example, it displays a window 252 that provides general information on the patient “R, Joseph.” It has another window 254 that provides a workspace for creating the final ECG report. Typically, these ECG reports are a set of specific codes, displayed in window 256 that correspond to different conditions.

FIG. 5 illustrates an exemplary draft report 258 as generated by a machine interpretation. It comprises a series of lines that correspond to different conditions. Typically, they are ordered in their relative importance. The physician, at the workstation, will review the specific ECG wave data and revise the draft report generated from the machine-generated interpretation. These series of statements 01-07 (280), providing specific diagnoses, will then be edited in order to generate the final report 126 that is stored in the patient database 130.

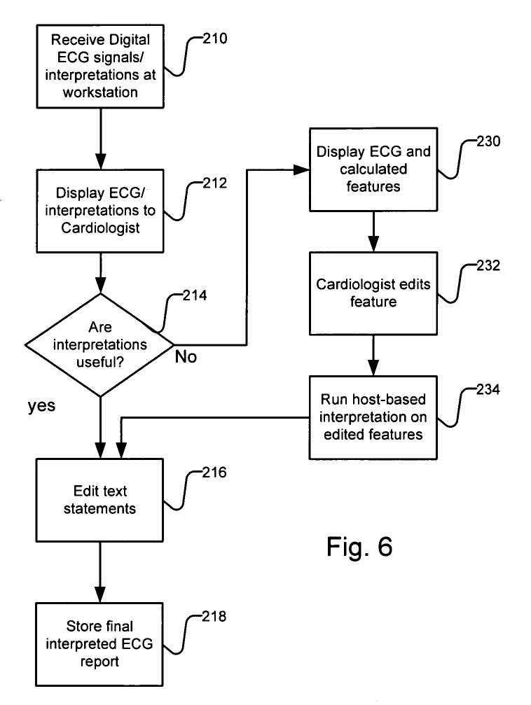

FIG. 6 illustrates a process for feature-leveling editing according to the present invention. Specifically, as in the past, the digital ECG data including the interpretation, are received at the workstation 124 in step 210 from the host 130. The ECG data including the interpretations are then displayed in step 212 to the cardiologist or other user/reader 122 on the workstation 124 using interfaces as illustrated in FIGS. 4 and 5.

According to the invention, then in step 214, the physician/cardiologist reviews the actual ECG wave data and determines whether or not the interpretations of the report are useful.

Most often, the interpretations will be largely correct and only require minor editing. In this case, the physician will simply edit the text statements in step 216 and then store the final ECG report in step 218 in the database 130.

However, in the situation where the interpretation is inaccurate, according to the invention, the workstation 124 displays the ECG data along with the calculated and otherwise determined features in step 230. The inventive feature-based editing permits the cardiologist or other reader/user to review and edit at the intermediate step of the machine-generated ECG interpretation. This is accomplished with a user interface that permits the display, review and editing of the measurements and features derived by the computer analysis (feature-based editing), along with the capability of resubmitting these features and measurements to the interpretive algorithms to generate a new list of interpretive statements.

Often, the features are as listed in Table I. According to the invention, the cardiologist or other user edits the specific values for the features. For example, one example may be the length of the QRS complex—in the situation where the interval was improperly measured by the algorithm. The physician manually edits feature or features in step 232. Then, the workstation 124 or host system 130 runs a host-based interpretation based on the cardiologist-edited features and the remaining calculated features in step 234. In one embodiment, the interpretation algorithm is a system licensed by the Glasgow Royal Infirmary. This generates a new ECG draft report, which the physician edits in step 216 for submission in step 218.

According to another implementation, the system also allows the option of selecting one or more specific pulses in the ECG wave data for analysis. As mentioned previously, features are typically pulled or calculated from a selected ECG wave from one typical cardiac cycle out of the usually very long wave data set that the machine acquires. Other systems calculated features base on an average ECG wave derived a series of waves to form the basis of the interpretation. Unfortunately, these processes do not always create the best basis for the analysis. Also, the cardiologist may want to force analysis of some other, atypical, for example, wave.

In one embodiment, as part of the feature selection process, the user/cardiologist is also provided with an opportunity to select a specific wave for analysis of a specific cardiac cycle. This addresses the problem of the system working from an atypical beat, sometimes referred to as a funny looking beat (FLB) or a beat initiated from pacemaker. On the other hand, the user can focus analysis on unusual beats such as beats from premature ventricular contractions (PVC), which are typically infrequent and will not be analyzed by conventional systems.

### EXAMPLE 1

The duration of one portion of the ECG wave, called the QRS complex, is used to determine if there is an interruption in the flow of electricity in the heart's conduction system. The QRS duration is a feature that directly leads to specific diagnoses on the ECG report. If the QRS duration feature is calculated to be greater than 120 milliseconds (and the QRS configuration matches a certain pattern), the condition is referred to as Left Bundle Branch Block, or “LBBB.” This means that the electricity is not conducting properly through the Left Bundle of the conducting system.

In the presence of LBBB on the ECG report, it is not possible to make any definitive statements about other clinical conditions such as anterior myocardial infarction (“AMI”) or left ventricular hypertrophy (“LVH”).

It is not uncommon for a previous ECG report for a patient to show a QRS duration of 118 milliseconds (below the cutoff for LBBB), along with an AMI and LVH. When the next ECG on that patient is measured to have a QRS duration of 120 milliseconds or greater, the reading will come out LBBB only.

The cardiologist reviewing the ECG may determine that the two ECGs are nearly identical, and that the pathology in the heart has not changed. On a conventional ECG editing system, for example, the cardiologist would have to change the QRS duration, delete the LBBB line, and add the LVH and AMI lines, resulting in excess effort and a time expenditure.

Using an embodiment of the present invention, it is only necessary to change the QRS duration to 118 milliseconds as in step 232 of FIG. 6. The interpretation is then regenerated using the specified QRS duration that would reflect the absence of LBBB, and the presence of AMI and LVH in step 234.

### EXAMPLE 2

P-waves are very tiny, and hard for the computer to distinguish on an ECG, especially if the patient is moving or other noise is present. The computer might also incorrectly think that P-waves are present, getting confused by the presence of noise. In this latter case, the reading might be “Sinus rhythm” and “1st degree AV block.” A measurement of the PR Interval will be reported (the time duration between the occurrence of the P-wave and the occurrence of the QRS complex).

In a statement-based editing environment, the cardiologist might change the reading of “sinus rhythm” to “atrial fibrillation.” If the reading is left this way, which is very common, there is a conflict. “1st degree AV block” means that the PR interval is longer than 200 milliseconds, but there is no PR interval if the is no P-wave.

The correct next step is for the cardiologist with convention editing systems is to remove measurement of the PR interval, and delete the line that says “1st degree AV block.” Here the cardiologist is performing a rather clerical function in the statement-based editing environment.

In an embodiment of the present invention, a feature-based editing environment is provided that enables the removal of feature data relating to the presence of the P-waves, specifically that the P-waves were found. Then, when the interpretation is re-run on the host, the reading will now automatically be changed to “atrial fibrillation,” the PR interval will be removed, and the statement “1st degree AV Block” will be removed. As a result, the cardiologist has been able to interact as a cardiologist, not as a clerk. Inconsistencies and mutually exclusive findings have been avoided in the reading.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for generating an electrocardiogram (ECG) report, the method comprising:
receiving ECG data at a physician workstation;
enabling a user to specify values for features of the ECG data;
generating an interpretation of the ECG data based on the values of the features specified by the user; and
storing the interpretation after possible editing in a patient database.

2. A method as claimed in claim 1, wherein the ECG data includes ECG wave data and a ECG machine interpretation of the ECG wave data.

3. A method as claimed in claim 2, further comprising generating the ECG wave data at a cart and the ECG machine interpretation at a host system.

4. A method as claimed in claim 1, wherein the user selects whether to edit the machine interpretation or specify values for the features.

5. A method as claimed in claim 1, wherein the step of enabling feature value specification comprises presenting machine generated values for features of the ECG wave data and enabling the user to specify different values for at least one of the features.

6. A method as claimed in claim 5, wherein the step of generating the interpretation comprises generating a re-interpretation of the ECG data using the specified values for the features and the machine-generated values for other features.

7. A system for generating an electrocardiogram (ECG) report, the system comprising:
a workstation that receives ECG data and enables specification of values for features of the ECG data;
an interpretation system for evaluating of the ECG data based on the values of the features specified by a user and generating an interpretation for storage in a patient database.

8. A system as claimed in claim 7, wherein the ECG data includes ECG wave data and a ECG machine interpretation of the ECG wave data.

9. A system as claimed in claim 8, further comprising an ECG cart for generating the ECG wave data and a host system for generating the ECG machine interpretation.

10. A system as claimed in claim 7, wherein the workstation allows the user to select whether to edit a machine interpretation f or specify values for the features.

11. A system as claimed in claim 7, wherein the workstation presents machine generated values for features of the ECG wave data and enables the user to specify different values for at least one of the features.

12. A system as claimed in claim 11, wherein the interpretation system generates an interpretation of the ECG data using the specified values for the features and the machine generated values for other features.

13. A computer software product for generating an electrocardiogram (ECG) report, the product comprising a computer-readable medium in which program instructions are stored, which instructions, when read by a computer, cause the computer to receive ECG data at a physician workstation, enable a user to specify values for features of the ECG data, generate an interpretation of the ECG data based on the values of the features specified by the user, and store the interpretation after possible editing in a patient database.

14. A product as claimed in claim 13, wherein the ECG data includes ECG wave data and a ECG machine interpretation of the ECG wave data.

15. A product as claimed in claim 14, wherein the instructions cause the computer to generate the ECG machine interpretation at a host system.

16. A product as claimed in claim 13, wherein the instructions cause the computer to enable the user to select whether to edit the machine interpretation or specify values for the features.

17. A product as claimed in claim 13, wherein the instructions cause the computer to enable feature value specification by presenting machine generated values for features of the ECG wave data and enabling the user to specify different values for at least one of the features.

18. A product as claimed in claim 13, wherein the instructions cause the computer to generate the interpretation by generating a re-interpretation of the ECG data using the specified values for the features and the machine-generated values for other features.
